(19) United States
(12) Patent Application Publication
(10) Pub. No.: US 2022/0098663 A1
(43) Pub. Date: Mar. 31, 2022

(54) BIOMARKERS FOR CONGENITAL VASCULAR MALFORMATIONS

(71) Applicant: University of South Carolina, Columbia, SC (US)

(72) Inventors: Wenbin Tan, Columbia, SC (US); Elaine G. Taine, Columbia, SC (US)

(73) Assignee: University of South Carolina, Columbia, SC (US)

(21) Appl. No.: 17/386,733

(22) Filed: Jul. 28, 2021

Related U.S. Application Data
(60) Provisional application No. 63/084,109, filed on Sep. 28, 2020.

(51) Int. Cl. C12Q 1/6876

(52) U.S. Cl. CPC C12Q 1/6876; C12Q 2600/112

(57) ABSTRACT

Described herein are biomarkers that have shown significantly increased expression levels in malformed blood vessels as compared to normal vessels, as well as methods of use employing the novel biomarkers to provide early diagnosis of congenital vascular malformations as well as treatment for same.

### FEDERALLY SPONSORED RESEARCH STATEMENT

This invention was made with government support under R01 AR073172 awarded by National Institute of Health. The government may have certain rights in the invention.

### TECHNICAL FIELD

The subject matter disclosed herein is generally directed to biomarkers that have shown significantly increased expression levels in malformed blood vessels as compared to normal vessels, as well as methods and assays employing the novel biomarkers to provide early diagnosis of congenital vascular malformations.

### BACKGROUND

Vascular malformations are abnormalities in the blood vessels, including veins, lymph vessels, capillaries, and arteries. They are usually congenital, meaning they are present at birth, although some appear in the days and weeks after birth. Some vascular malformations—such as a hemangioma, also known as a strawberry mark-are visible on the skin. They are not dangerous and may require cosmetic treatment or no treatment at all. Other vascular malformations, such as arteriovenous malformations and venous malformations, are located inside the body. Some internal malformations have a corresponding sore or mark on the skin.

If an internal malformation grows unchecked, it can cause painful compression of nerves and organs; hemorrhage, or internal bleeding; and necrosis, or tissue death. Depending on the location of a vascular malformation and the structures it affects, it can also cause constipation, pelvic pressure, or swelling in the limbs.

Arteriovenous malformations, commonly known as AVMs, are abnormal tangles or clusters of blood vessels that can form wherever arteries and veins are located. In an arteriovenous malformation, the capillaries—the tiny vessels that carry blood from arteries to veins and supply blood to the tissues—are missing. This causes a kind of “short circuit” in a person's circulation, putting increased pressure on the arteries and veins and potentially weakening them over time.

Depending on their location, arteriovenous malformations can cause bleeding or organ compression. For instance, a malformation in the pelvis can cause bleeding in the uterus or bladder. A malformation in the abdomen may cause intestinal bleeding. If located in the chest, it can stress the heart.

Some arteriovenous malformations require treatment to prevent compression of nearby organs or to manage bleeding. For others, our doctors recommend monitoring without immediate treatment, called watchful waiting.

Venous malformations are the most common type of vascular malformation. They are caused by abnormal widening of the veins or a tangle of small veins that does not affect the arteries. Venous malformations can occur near the surface of the skin or deep inside the body. If located near the surface, these malformations cause a lump beneath the skin and a corresponding bluish birthmark or skin lesion that is present at birth and becomes larger as a child grows. Skin lesions caused by a venous malformation can be painful. If the skin that covers the malformation is very thin and becomes stretched as it grows, it can bleed. Blood can pool in the dilated veins, resulting in painful, but not dangerous, blood clots, known as thrombophlebitis.

Venous malformations often occur in people with Klippel-Trénaunay syndrome, a rare congenital condition that affects the development of blood vessels, soft tissues, and bones. Many people with this condition are born with a dark red birthmark called a port wine stain, which is caused by swelling in the capillaries near the surface of the skin.

The overall incidence of congenital vascular malformations in the general population is about 1.5%, which is about 4.8 million patients in the U.S. and 105.5 million patients worldwide as the direct targeting population.

Given that congenital vascular malformations may have long-lasting and potentially harmful effects on patients, improved systems and methods for early discovery of the malformations are needed and disclosed herein.

### SUMMARY

The above objectives are accomplished according to the present disclosure by providing in one embodiment, a method for screening for congenital vascular anomalies. The method may include obtaining a biological sample, screening the biological sample for at least one biomarker indicative of at least one congenital vascular malformation, and the at least one biomarker has a genetic sequence identified in SEQ. ID. NOS. 1-5. Further, the method may employ next-generation sequencing, FISH analysis, immunohistochemistry, or a liquid biopsy to determine the presence of the at least one biomarker in the biological sample. Yet still, the biological sample may be obtained from a lesional tissue, serum, circulating exosomes, or body fluids. Again, the at least one congenital vascular malformation may include port wine stain. Still yet, the congenital vascular malformation may be symptomatic or asymptomatic. Further again, screening may be administered prior to vascular lesions growing to a detectable size via diagnostic radiology. Further yet, the congenital vascular malformation is visible or invisible.

In a further embodiment, the disclosure may provide a molecular assay for early diagnosis of congenital vascular malformations. The assay may include obtaining a biological sample, screening the biological sample for at least one biomarker indicative of at least one congenital vascular malformation, wherein the at least one biomarker has a genetic sequence identified in SEQ. ID. NOS. 1-5, and the molecular assay may be administered prior to vascular lesions growing to a size detectable by diagnostic radiology. Still, the assay may include employing next-generation sequencing, FISH analysis, immunohistochemistry, or a liquid biopsy to determine the presence of the at least one biomarker in the biological sample. Further, the biological sample may be obtained from a lesional tissue, serum, circulating exosomes, or body fluids. Still again, the at least one congenital vascular malformation may comprise port wine stain. Yet again, the congenital vascular malformation may be symptomatic or asymptomatic. Furthermore, the congenital vascular malformation may be visible or invisible. Still yet, the assay may be administered to a new born or early childhood aged children.

These and other aspects, objects, features, and advantages of the example embodiments will become apparent to those having ordinary skill in the art upon consideration of the following detailed description of example embodiments.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### GENERAL DEFINITIONS

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

The term “optional” or “optionally” means that the subsequent described event, circumstance or substituent may or may not occur, and that the description includes instances where the event or circumstance occurs and instances where it does not.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, as used herein, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

Biological markers (biomarkers) may be defined cellular, biochemical or molecular alterations that are measurable in biological media such as human tissues, cells, or fluids. More recently, the definition has been broadened to include biological characteristics that can be objectively measured and evaluated as an indicator of normal biological processes, pathogenic processes, or pharmacological responses to a therapeutic intervention.

In practice, biomarkers include tools and technologies that can aid in understanding the prediction, cause, diagnosis, progression, regression, or outcome of treatment of disease. These may involve measurements directly on biological media (e.g., blood or cerebrospinal fluid) or measurements such as brain imaging which do not involve direct sampling of biological media but measure changes in the composition or function of tissues, organs, systems, etc.

Biomarkers of all types have been used by epidemiologists, physicians, and scientists to study human disease. The application of biomarkers in the diagnosis and management of cardiovascular disease, infections, immunological and genetic disorders, and cancer are well known. Their use in research has grown out of the need to have a more direct measurement of exposures in the causal pathway of disease that is free from recall bias, and that can also have the potential of providing information on the absorption and metabolism of the exposures. Neuroscientists have also relied on biomarkers to assist in the diagnosis and treatment of nervous system disorders and to investigate their cause. Blood, brain, cerebrospinal fluid, muscle, nerve, skin, and urine may be employed to gain information about patients in both the healthy and diseased states.

There are two major types of biomarkers: biomarkers of exposure, which are used in risk prediction, and biomarkers of disease, which are used in screening and diagnosis and monitoring of disease progression. Biomarkers used in risk prediction, in screening, and as diagnostic tests are well established, and they offer distinct and obvious advantages. The classification of many diseases is based on either standardized clinical criteria or histological diagnoses. Biomarkers also have the potential to identify disease at an early stage, to provide a method for homogeneous classification of a disease, and to extend the knowledge base concerning the underlying disease pathogenesis. These advantages have direct application to all types of clinical investigation, from clinical trials to observational studies in epidemiology.

In epidemiological (or quasi-experimental) investigations, biomarkers improve validity while reducing bias in the measurement of exposures (or risk factors) for disease. Rather than relying on a history of exposure to a putative risk factor, direct measurement of the level of exposure or the chromosomal alteration resulting from the exposure lessens the possibility of misclassification of exposure. Such misclassifications not only produce inaccurate and deceptive results but also reduce the power of studies to detect health effects. Thus, the use of biomarkers improves the sensitivity and specificity of the measurement of the exposures or risk factors.

Molecular biomarkers have the additional potential to identify individuals susceptible to disease. Molecular genetics have already had an impact on neurological practice, leading to improved diagnosis. Classification of populations in terms of the degree of susceptibility on the basis of such biomarkers produces greater accuracy than relying on historical definitions of susceptibility. For example, a biomarker will allow the stratification of a population on the basis of a specific genotype associated with a disease rather than relying on a report of family history of the disease. The ability to quantify susceptibility in this way can be an extremely important method for estimating disease risk among various populations.

Biomarkers depicting prodromal signs enable earlier diagnosis or allow for the outcome of interest to be determined at a more primitive stage of disease. Blood, urine, and cerebrospinal fluid may provide the necessary biological information for the diagnosis. In these conditions, biomarkers are used as an indicator of a biological factor that represents either a subclinical manifestation, stage of the disorder, or a surrogate manifestation of the disease. Biomarkers used for screening or diagnosis also often represent surrogate manifestations of the disease.

The use of this class of biomarkers include: 1) identification of individuals destined to become affected or who are in the “preclinical” stages of the illness, 2) reduction in disease heterogeneity in clinical trials or epidemiologic studies, 3) reflection of the natural history of disease encompassing the phases of induction, latency and detection, and 4) target for a clinical trial. The improvement in validity and precision far outweigh the difficulty in obtaining such tissues from patients.

Diagnostic tests for diseases are used with increased frequency in clinical research and practice. In the diagnostic effort, collection of information from various sources, some of which includes results from diagnostic tests, helps to achieve the ultimate goal of increasing the probability of a given diagnosis. Clinical tests are also performed, though probably less often, for other reasons such as the following: to measure disease severity, to predict disease occurrence, or to monitor the response to a particular treatment. More importantly, biomarkers for disease easily lend themselves to clinical trials. Another advantage of this type of diagnostic test is the reduction in disease heterogeneity in clinical trials or observational epidemiologic studies, leading to better understanding of natural history of disease encompassing the phases of induction, latency and detection.

Precise numbers are enticing, but they are prone to the same problems as any variable. Reliability, validity, sensitivity, specificity, ascertainment bias, and interpretation of data using biomarkers should be reviewed just as carefully as any other variable. These problems remain whether the biomarker is being used as a variable in a clinical trial or in an epidemiologic study.

The evaluation of the validity of a biomarker is complex and may include a host of factors including: 1) content validity, which shows the degree to which a biomarker reflects the biological phenomenon studied; 2) construct validity, which pertains to other relevant characteristics of the disease or trait, for example other biomarkers or disease manifestations; and, 3) criterion validity, which shows the extent to which the biomarker correlates with the specific disease and is usually measured by sensitivity, specificity, and predictive power. To further evaluate the effect of misclassification of disease, false positives and false negatives as well as positive and negative predictive power should also be estimated. In an ideal situation the biomarker has a clear predictive value, such as those disclosed herein. The use of receiver-operator characteristic curves can provide the tools necessary to determine the best choice in terms of sensitivity and false-positive rates, particularly when other tests are used.

Most would agree that screening tests would be very desirable for chronic progressive disorders. One purpose of screening is early detection with the hope of preventing the illness altogether. Many of the methods and concerns related to diagnostic testing apply to screening as well. As with other diagnostic methods, sensitivity and specificity tell us the accuracy of the test but not the probability of disease. For that we need to estimate the predictive values (positive and negative). Positive predictive value (PPV) is the percentage of people with a positive test who actually have the disease. This provides us with information about the likelihood of the disease being present if the test is positive. Negative predictive value (NPV) is the percentage of people with a negative test who do not have the disease. Increasing the prior probability will increase the PPV but decrease the NPV, assuming that the sensitivity and specificity remain unchanged. Similar changes in the predictive values occur with changes in the prevalence of a condition as will be discussed in screening.

Since validity is measured by sensitivity and specificity and predictive power by PPV and NPV, a major difference in evaluating screening and diagnostic tests is the pretest probability. Screening, by definition, includes a larger number of individuals without the disease, generally ascertained via a defined population sample. Diagnostic tests are designed to improve clinical diagnoses by enhancing the probability of disease, and by definition the pretest probability would be high. However, for screening the prior probability is much lower and that effect will lower the PPV. Therefore, screening also requires careful consideration of prevalence, or the prior probability of disease. These analytic methods are now available on many software statistical packages.

The investigator must be clear about the use of the biomarker. Errors are most often made when biomarker data are over interpreted. For example, the results of one study may indicate that a specific biomarker (collected as a measure of an exposure or susceptibility) is strongly associated with a particular disease or outcome. The investigator, on the other hand, interprets the result as a biomarker for the disease or the observed outcome. No matter how high the odds ratio or relative risk, a biomarker of this type could not be expected to function as a diagnostic test unless it is a manifestation of the disease. For example, the APOE-ε4 allele is strongly associated with Alzheimer's disease, but its presence does not infer disease. Many patients without an APOE-ε4 allele develop Alzheimer's disease and some individuals with an APOE-ε4 allele do not develop this condition.

Advantages of employing biomarkers include they provide: an objective assessment; precision of measurement; reliability, validity can be established; they are less biased than questionnaires; disease mechanisms are often studied; and homogeneity of risk or disease.

Biomarkers can serve as early warning systems for health. For example, high levels of lead in the bloodstream may indicate a need to test for nervous system and cognitive disorders, especially in children. High cholesterol levels are a common biomarker for heart disease risk.

Many biomarkers come from simple measurements made during a routine doctor visit, like blood pressure or body weight. Other biomarkers are based on laboratory tests of blood, urine, or tissues. Some capture changes at the molecular and cellular level by looking at genes or proteins.

Biomarkers play an important role in illuminating relationships among environmental exposures, human biology, and disease. Scientists can use biomarkers to better understand fundamental biological processes, advance exposure science, and turn research findings into practical medical and public health applications.

Various ways of testing samples for biomarkers exist. These may include, but are not limited to high-capacity, high-throughput GLP/GCP bioanalysis for small molecules, non-GLP discovery bioanalysis, drug-drug interaction (DDI), ADME, PK, toxicology, bioequivalence studies, LC-MS/MS (triple quadrupole and QTRAP), UHPLC and HPLC, conventional HPLC with UV, fluorescence, conductivity, electrochemical detection, comprehensive next-generation sequencing (NGS), FISH analysis, Immunohistochemistry uses proteins called antibodies to locate markers in the tissue sample, liquid biopsies such as Guardant360 CDx and FoundationOne Liquid CDx, etc. One may test for biomarkers in blood, urine, stool, tissues, or other bodily fluids.

Biomarkers differ from psychometric measures in at least four major ways. Most importantly, they do not rely on valid self-reporting, and, hence, are not vulnerable to problems of inaccurate recall or reluctance of individuals to give candid reports of their behaviors or attitudes. They can thus add credibility to research dealing with treatment efficacy and can provide clinicians with an additional source of objective information on patients.

Second, although biomarkers are subject to many of the usual psychometric issues of validity and reliability, some, such as internal consistency and construct validity, are not relevant to their evaluation. Instead, major concerns in evaluating biomarkers deal with criterion validity, stability, test-retest consistency, and interrater reliability. These issues have a bearing particularly for new markers for which fully automated test procedures have yet to be developed.

Third, the expertise required to ensure valid results from biomarkers is somewhat different from that needed to obtain maximally valid self-report information, where rapport, assurance of confidentiality, motivation for honesty, current state, and testing conditions are important considerations. The accuracy of biomarker information is rarely a function of sample collection, but rather is closely related to sample handling, storage, and transmittal; quality assurance of laboratory procedures for isolation of the biomarker; and methods for quantifying and interpreting results.

Finally, strictly speaking, biomarkers are reflections of physiological reactions. Self-report screening scales, on the other hand, generally use a diagnosis of a condition as the criterion against which they are evaluated. Assessment of behavior per se and severity of disease are both important, albeit somewhat non-overlapping phenomena.

The present disclosure builds on work as far back as 2015, when the inventors discovered: (1) a panel of molecules including IGFBP5, ADAM30, MMP12, HIF1a, etc., have increased expression levels in Port Wine Stain (PWS) blood vessels; and (2) there is a collagenous hypertrophy in infantile and early childhood PWS lesions.

In 2017, the inventors characterized expression patterns of various types of Collagens in PWS vasculature from patients by immunoblot and identified upregulation of Collagen V, VI, VIII, XVI, XXII, XXIII, and XXV in patient biopsy samples.

From September 2017 to November 2018, the inventors further identified the expression pattern of collagen V in patients' blood vessels by IHC. These upregulated collagens can be bio-markers for diagnostics of vascular malformations. The intervention of collagens with vascular targeting treatment by Pulsed Dye Laser (“PDL”), in particular in children, will result in a better outcome than PDL only.

Congenital vascular malformations present prenatally and at birth, visible or invisible. They can be symptomatic or asymptomatic, causing morbidity and pain. Therefore, early detection is of importance for the management.

The inventors have found that a panel of molecules, including ADAM30, IGFBP5, MMP12, Collagen V, VIII and XXIII, etc., showed significantly increased expression levels in those malformed blood vessels than normal ones. These molecules can serve as novel biomarkers to help early diagnosis of congenital vascular malformations. These biomarkers can be found in either lesional tissues, serum or circulating exosomes or other body fluids. In addition, the inventors found Src is the crucial kinase that have been activated in malformed blood vessels; therefore, Src inhibitors can be used for the treatment for these disorders. Src Inhibitor-1 is a member of the class of quinazolines that is quinazoline which is substituted at position 4 by a p-phenoxyanilino group and at positions 6 and 7 by methoxy groups. It is a potent, competitive dual site (both the ATP- and peptide-binding) Src kinase inhibitor. Src Inhibitor-1 is one of the ‘gold standards’ for Src kinase inhibition that has been shown to use PP1 or PP2 in parallel with Src-I1 to inhbit Src family kinases. It has a role as an EC 2.7.10.2 (non-specific protein-tyrosine kinase) inhibitor. It is a member of quinazolines, a secondary amino compound, an aromatic ether and a polyether.

Src is the cellular counterpart of the first identified viral oncogene v-Src. It forms part of a large family of nonreceptor tyrosine kinases that have been extensively studied over the last few decades. This has led to the realization that Src can regulate a number of signaling pathways that impact on the behavior of tumor cells, including proliferation, survival, migration, invasion, and angiogenesis. There are various Src inhibitors (dasatinib, saracatinib, bosutinib, KX01, etc.) that may be employed herein.

This inventors discovered that a set of molecules such as ADAM30, IGFBP5, MMP 12, Collagen V, VIII and XXIII, etc. have increased in blood vessels of congenital vascular malformations. The increases in these molecules can be identified from patients' serum. The assay developed to detect these molecules from patients serum can help early diagnosis of these types of diseases. These diagnostic biomarkers are particularly valuable for infantile and early childhood patients whose vascular lesions in brain or other internal organs are unable to be detected by CT or MRI during the early stages of the diseases.

The current disclosure provides many potential applications. First, screening for congenital vascular anomalies for subjects who are unaware of existence of vascular anomalies in internal organs. Second, early diagnosis of congenital vascular anomalies for infantile and early childhood patients whose vascular lesions in brain or other internal organs are unable to be detected by CT or MRI during the early stages of the diseases. Third, target lesional blood vessels in combination with collagen hypertrophy in patients, particularly in infantile and early childhood patients, to provide better outcomes than targeting lesional blood vessels alone. Further, congenital vascular malformations present prenatally and at birth, visible or invisible. They can be symptomatic or asymptomatic, causing morbidity and pain. Therefore, early detection is of importance for the management.

There is currently no molecular assay for early diagnosis of congenital vascular malformations available in the market. Diagnostic radiology, such as CT, MRI, angiography or Doppler scanning, are the current diagnostic methods used in clinic. However, these methods are not capable of early diagnosis until vascular lesions grow to certain detectable sizes nor screen the subjects with potential risks of existence of vascular anomalies in internal organs in a large population due to high costs. In addition, the current disclosure can potentially be used for every new born or early childhood children as a screening assay for the disease. The current disclosure provides the first molecular diagnostic assay for congenital vascular malformations. Early detection and early awareness of vascular lesions are important, as this will affect management strategies.

SEQUENCE LISTING - USC 2033101.000185

<110> University of South Carolina
<120> BIOMARKERS FOR CONGENITAL VASCULAR
MALFORMATIONS
<130> 2033101.0000304
<140> Unknown
<141> 2021-07-28
<150> U.S. Provisional Application No. 63/084,109
<151> 09-28-2020
<160> 6
<170> PatentIn
<210> 1
<211> 790
<212> PRT
<213> Homo sapiens
<221> CDS
<222> 1 ... 790
<400> 1
MRSVQIFLSQ CRLLLLLVPT MLLKSLGEDV IFHPEGEFDS
YEVTIPEKLS FRGEVQGVVS PVSYLLQLKG KKHVLHLWPK RLLLPRHLRV
FSFTEHGELL EDHPYIPKDC NYMGSVKESL DSKATISTCM GGLRGVFNID
AKHYQIEPLK ASPSFEHVVY LLKKEQFGNQ VCGLSDDEIE WQMAPYENKA
RLRDFPGSYK HPKYLELILL FDQSRYRFVN NNLSQVIHDA ILLTGIMDTY
FQDVRMRIHL KALEVWTDFN KIRVGYPELA EVLGRFVIYK KSVLNARLSS
DWAHLYLQRK YNDALAWSFG KVCSLEYAGS VSTLLDTNIL APATWSAHEL
GHAVGMSHDE QYCQCRGRLN CIMGSGRTGF SNCSYISFFK HISSGATCLN
NIPGLGYVLK RCGNKIVEDN EECDCGSTEE CQKDRCCQSN CKLQPGANCS
IGLCCHDCRF RPSGYVCRQE GNECDLAEYC DGNSSSCPND VYKQDGTPCK
YEGRCFRKGC RSRYMQCQSI FGPDAMEAPS ECYDAVNLIG DQFGNCEITG
IRNFKKCESA NSICGRLQCI NVETIPDLPE HTTIISTHLQ AENLMCWGTG
YHLSMKPMGI PDLGMINDGT SCGEGRVCFK KNCVNSSVLQ FDCLPEKCNT
RGVCNNRKNC HCMYGWAPPF CEEVGYGGSI DSGPPGLLRG AIPSSIWVVS
IIMFRLILLI LSVVFVFFRQ VIGNHLKPKQ EKMPLSKAKT EQEESKTKTV
QEESKTKTGQ EESEAKTGQE ESKAKTGQEE SKANIESKRP KAKSVKKQKK
<210> 2
<211> 233
<212> PRT
<213> Homo sapiens
<222> 1-233
<400> 2
MVLLTAVLLL LAAYAGPAQS LGSFVHCEPC DEKALSMCPP
SPLGCELVKE PGCGCCMTCA LAEGQSCGVY TERCAQGLRC LPRQDEEKPL
HALLHGRGVC LNEKSYREQV KIGCAPGGQR HICFSLYYSP HPKLPSPGLW
PCAPWPTGAD PPAPSPAERD SREHEEPTTS EMAEETYSPK IFRPKHTRIS
ELKAEAVKKD RRKKLTQSKF VGGAENTAHP RIISAPEMRQ ESE
<210> 3
<211> 470
<212> PRT
<213> Homo sapiens
<222> 1-470
<400> 3
MKFLLILLLQATASGALPLNSSTSLEKNNVLFGERYLEKFYGLEINKLP
VTKMKYSGNLMKEKIQEMQHFLGLKVTGQLDTSTLEMMHAPRCGVPDVHHFR
EMPGGPVWRKHYITYRINNYTPDMNREDVDYAIRKAFQVWSNVTPLKFSKINT
GMADILVVFARGAHGDFHAFDGKGGILAHAFGPGSGIGGDAHFDEDEFWTTHS
GGTNLFLTAVHEIGHSLGLGHSSDPKAVMFPTYKYVDINTFRLSADDIRGIQSLY
GDPKENQRLPNPDNSEPALCDPNLSFDAVTTVGNKIFFFKDRFFWLKVSERPKT
SVNLISSLWPTLPSGIEAAYEIEARNQVFLFKDDKYWLISNLRPEPNYPKSIHSFG
FPNFVKKIDAAVFNPRFYRTYFFVDNQYWRYDERRQMMDPGYPKLITKNFQGI
GPKIDAVFYSKNKYYYFFQGSNQFEYDFLLQRITKTLKSNSWFGC
<2 10> 4
<211> 1837
<212> PRT
<213> Homo sapiens
<222> 1-1837
MDVHTRWKAR SALRPGAPLL PPLLLLLLWA PPPSRAAQPA
DLLKVLDFHN LPDGITKTTG FCATRRSSKG PDVAYRVTKD AQLSAPTKQL
YPASAFPEDF SILTTVKAKK GSQAFLVSIY NEQGIQQIGL ELGRSPVFLY
EDHTGKPGPE DYPLFRGINL SDGKWHRIAL SVHKKNVTLILDCKKKTTKF
LDRSDHPMID INGIIVFGTR ILDEEVFEGD IQQLLFVSDH
RAAYDYCEHYSPDCDTAVPD TPQSQDPNPD EYYTEGDGEG ETYYYEYPYY
EDPEDLGKEP TPSKKPVEAAKETTEVPEEL TPTPTEAAPM PETSEGAGKE
EDVGIGDYDY VPSEDYYTPS PYDDLTYGEG EENPDQPTDP GAGAEIPTST
ADTSNSSNPA PPPGEGADDL EGEFTEETIR NLDENYYDPYYDPTSSPSEI
GPGMPANQDT IYEGIGGPRG EKGQKGEPAI IEPGMLIEGP
PGPEGPAGLPGPPGTMGPTG QVGDPGERGP PGRPGLPGAD GLPGPPGTML
MLPFRFGGGG DAGSKGPMVS AQESQAQAIL QQARLALRGP AGPMGLTGRP
GPVGPPGSGG LKGEPGDVGP QGPRGVQGPP GPAGKPGRRG RAGSDGARGM
PGQTGPKGDR GFDGLAGLPG EKGHRGDPGP SGPPGPPGDD GERGDDGEVG
PRGLPGEPGP RGLLGPKGPP GPPGPPGVTG MDGQPGPKGN VGPQGEPGPP
GQQGNPGAQG LPGPQGAIGP PGEKGPLGKP GLPGMPGADG PPGHPGKEGP
PGEKGGQGPP GPQGPIGYPG PRGVKGADGI RGLKGTKGEK GEDGFPGFKG
DMGIKGDRGE IGPPGPRGED GPEGPKGRGG PNGDPGPLGP PGEKGKLGVP
GLPGYPGRQG PKGSIGFPGF PGANGEKGGR GTPGKPGPRG QRGPTGPRGE
RGPRGITGKP GPKGNSGGDG PAGPPGERGP NGPQGPTGFP GPKGPPGPPG
KDGLPGHPGQ RGETGFQGKT GPPGPPGVVG PQGPTGETGP MGERGHPGPP
GPPGEQGLPG LAGKEGTKGD PGPAGLPGKD GPPGLRGFPG DRGLPGPVGA
LGLKGNEGPP GPPGPAGSPG ERGPAGAAGP IGIPGRPGPQ GPPGPAGEKG
APGEKGPQGP AGRDGLQGPV GLPGPAGPVG PPGEDGDKGE IGEPGQKGSK
GDKGEQGPPG PTGPQGPIGQ PGPSGADGEP GPRGQQGLFG QKGDEGPRGF
PGPPGPVGLQ GLPGPPGEKG ETGDVGQMGP PGPPGPRGPS GAPGADGPQG
PPGGIGNPGA VGEKGEPGEA GEPGLPGEGG PPGPKGERGE KGESGPSGAA
GPPGPKGPPG DDGPKGSPGP VGFPGDPGPP GEPGPAGQDG PPGDKGDDGE
PGQTGSPGPT EPGPSGPPG KRGPPGPAGP EGRQGEKGAK GEAGLEGPPG
KTGPIGPQGA PGKPGPDGLR GIPGPVGEQG LPGSPGPDGP PGPMGPPGLP
GLKGDSGPKG EKGHPGLIGL IGPPGEQGEK GDRGLPGPQG SSGPKGEQGI
TGPSGPIGPP GPPGLPGPPG PKGAKGSSGP TGPKGEAGHP GPPGPPGPPG
EVIQPLPIQA SRTRRNIDAS QLLDDGNGEN YVDYADGMEE IFGSLNSLKL
EIEQMKRPLG TQQNPARTCK DLQLCHPDFP DGEYWVDPNQ GCSRDSFKVY
CNFTAGGSTC VFPDKKSEGA RITSWPKENP GSWFSEFKRG KLLSYVDAEG
NPVGVVQMTF LRLLSASAHQ NVTYHCYQSV AWQDAATGSY DKALRFLGSN
DEEMSYDNNP YIRALVDGCA TKKGYQKTVL EIDTPKVEQV PIVDIMFNDF
GEASQKFGFE VGPACFMG
<210>5
<211>887
<212> PRT
<213> Homo sapiens
<222> 1-887
EFRELGPGQG SVLLRDLEP GTDYEVTVSTL FGRSVGPATS
LMARTDASVE QTLRPVILGP TSILLSWNLV PEARGYRLEW RRETGLEPPQ
KVVLPSDVTR YQLDGLQPGT EYRLTLYTLL EGHEVATPAT VVPTGPELPV
SPVTDLQATD VPGQRVRVSW SPVPGATQYR IIWRSTQGVE RTLVLPGSQT
AFDLDDVQAG LSYTVRVSAR VGPREGSASV LTVRREPETP LAVPGLRVVV
SDATRVRVAW GPVPGASGFR ISWSTGSGPE SSQTLPPDST ATDITGLQPG
TTYQVAVSVL RGREEGPAAV IVARTDPLGP VRTVHVTQAS SSSVTITWTR
VPGATGYRVS WHSAHGPEKS QLVSGEATVA ELDGLEPDTE YTVHVRAHVA
GVDGPPASVV VRTAPEPVGR VSRLQILNAS SDVLRITWVG VTGATAYRLA
WGRSEGGPMR HQILPGNTDS AEIRGLEGGV SYSVRVTALV GDREGTPVSI
VVTTPPEAPP ALGTLHVVQR GEHSLRLRWE PVPRAQGFLL HWQPEGGQEQ
SRVLGPELSS YHLDGLEPAT QYRVRLSVLG PAGEGPSAEV TARTESPRVP
SIELRVVDTS IDSVTLAWTP VSRASSYILS WRPLRGPGQE VPGSPQTLPG
ISSSQRVTGL EPGVSYIFSL TPVLDGVRGP EASVTQTPVC PRGLADVVFL
PHATQDNAHR AEATRRVLER LVLALGPLGP QAVQVGLLSY SHRPSPLFPL
NGSHDLGIIL QRIRDMPYMD PSGNNLGTAV VTAHRYMLAP DAPGRRQHVP
GVMVLLVDEP LRGDIFSPIR EAQASGLNVV MLGMAGADPE QLRRLAPGMD
SVQTFFAVDD GPSLDQAVSG LATALCQASF TTQPRPEPCP VYCPKGQ
<210>6
<211>540
<212> PRT
<213> Homo sapiens
<222> 1-540
MGPGERAGGG GDAGKGNAAG GGGGGRSATT AGSRAVSALC
LLLSVGSAAA CLLLGVQAAA LQGRVAALEE ERELLRRAGP PGALDAWAEP
HLERLLREKL DGLAKIRTAR EAPSECVCPP GPPGRRGKPG RRGDPGPPGQ
SGRDGYPGPL GLDGKPGLPG PKGEKGAPGD FGPRGDQGQD GAAGPPGPPG
PPGARGPPGD TGKDGPRGAQ GPAGPKGEPG QDGEMGPKGP PGPKGEPGVP
GKKGDDGTPS QPGPPGPKGE PGSMGPRGEN GVDGAPGPKG EPGHRGTDGA
AGPRGAPGLK GEQGDTVVID YDGRILDALK GPPGPQGPPG PPGIPGAKGE
LGLPGAPGID GEKGPKGQKG DPGEPGPAGL KGEAGEMGLS GLPGADGLKG
EKGESASDSL QESLAQLIVE PGPPGPPGPP GPMGLQGIQG PKGLDGAKGE
KGASGERGPS GLPGPVGPPG LIGLPGTKGE KGRPGEPGLD GFPGPRGEKG
DRSERGEKGE RGVPGRKGVK GQKGEPGPPG LDQPCPVGPD GLPVPGCWHK

In some embodiments, a combination kit is provided that also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for screening for congenital vascular anomalies comprising;
obtaining a biological sample;
screening the biological sample for at least one biomarker indicative of at least one congenital vascular malformation; and
wherein the at least one biomarker has a genetic sequence identified in SEQ. ID. NOS. 1-6.

2. The method of claim 1, further comprising employing next-generation sequencing, FISH analysis, immunohistochemistry, or a liquid biopsy to determine the presence of the at least one biomarker in the biological sample.

3. The method of claim 1, wherein the biological sample is obtained from a lesional tissue, serum, circulating exosomes, or body fluids.

4. The method of claim 1, wherein the at least one congenital vascular malformation comprises port wine stain.

5. The method of claim 1, wherein the at least one congenital vascular malformation is symptomatic or asymptomatic.

6. The method of claim 1, wherein screening is administered prior to vascular lesions growing to a detectable size via diagnostic radiology.

7. The method of claim 1, wherein the at least one congenital vascular malformation is visible or invisible.

8. A molecular assay for early diagnosis of congenital vascular malformations comprising;
obtaining a biological sample;
screening the biological sample for at least one biomarker indicative of at least one congenital vascular malformation;
wherein the at least one biomarker has a genetic sequence identified in SEQ. ID. NOS. 1-6; and
wherein the molecular assay is administered prior to vascular lesions growing to a size detectable by diagnostic radiology.

9. The assay of claim 8, further comprising employing next-generation sequencing, FISH analysis, immunohistochemistry, or a liquid biopsy to determine the presence of the at least one biomarker in the biological sample.

10. The assay of claim 8, wherein the biological sample is obtained from a lesional tissue, serum, circulating exosomes, or body fluids.

11. The assay of claim 8, wherein the at least one congenital vascular malformation comprises port wine stain.

12. The assay of claim 8, wherein the at least one congenital vascular malformation is symptomatic or asymptomatic.

13. The assay of claim 8, wherein the at least one congenital vascular malformation is visible or invisible.

14. The assay of claim 8, wherein the assay is administered to a new born or early childhood aged children.


SEQUENCE LISTING (6 sequences)

SEQ ID NO: 1 - protein, 790 aa, Homo sapiens
MRSVQIFLSQCRLLLLLVPTMLLKSLGEDVIFHPEGEFDSYEVTIPEKLSFRGEVQGVVSPVSYLLQLKGKKHVLHLWPKRLLLPRHLRVFSFTEHGELLEDHPYIPKDCNYMGSVKESLDSKATISTCMGGLRGVFNIDAKHYQIEPLKASPSFEHVVYLLKKEQFGNQVCGLSDDEIEWQMAPYENKARLRDFPGSYKHPKYLELILLFDQSRYRFVNNNLSQVIHDAILLTGIMDTYFQDVRMRIHLKALEVWTDFNKIRVGYPELAEVLGRFVIYKKSVLNARLSSDWAHLYLQRKYNDALAWSFGKVCSLEYAGSVSTLLDTNILAPATWSAHELGHAVGMSHDEQYCQCRGRLNCIMGSGRTGFSNCSYISFFKHISSGATCLNNIPGLGYVLKRCGNKIVEDNEECDCGSTEECQKDRCCQSNCKLQPGANCSIGLCCHDCRFRPSGYVCRQEGNECDLAEYCDGNSSSCPNDVYKQDGTPCKYEGRCFRKGCRSRYMQCQSIFGPDAMEAPSECYDAVNLIGDQFGNCEITGIRNFKKCESANSICGRLQCINVETIPDLPEHTTIISTHLQAENLMCWGTGYHLSMKPMGIPDLGMINDGTSCGEGRVCFKKNCVNSSVLQFDCLPEKCNTRGVCNNRKNCHCMYGWAPPFCEEVGYGGSIDSGPPGLLRGAIPSSIWVVSIIMFRLILLILSVVFVFFRQVIGNHLKPKQEKMPLSKAKTEQEESKTKTVQEESKTKTGQEESEAKTGQEESKAKTGQEESKANIESKRPKAKSVKKQKK

SEQ ID NO: 2 - protein, 233 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIGCAPGGQRHICFSLYYSPHPKLPSPGLWPCAPWPTGADPPAPSPAERDSREHEEPTTSEMAEETYSPKIFRPKHTRISELKAEAVKKDRRKKLTQSKFVGGAENTAHPRIISAPEMRQESE

SEQ ID NO: 3 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 4 - protein, 1837 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 5 - protein, 887 aa, Homo sapiens
EFRELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATDVPGQRVRVSWSPVPGATQYRIIWRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQ

SEQ ID NO: 6 - protein, 540 aa, Homo sapiens
MGPGERAGGGGDAGKGNAAGGGGGGRSATTAGSRAVSALCLLLSVGSAAACLLLGVQAAALQGRVAALEEERELLRRAGPPGALDAWAEPHLERLLREKLDGLAKIRTAREAPSECVCPPGPPGRRGKPGRRGDPGPPGQSGRDGYPGPLGLDGKPGLPGPKGEKGAPGDFGPRGDQGQDGAAGPPGPPGPPGARGPPGDTGKDGPRGAQGPAGPKGEPGQDGEMGPKGPPGPKGEPGVPGKKGDDGTPSQPGPPGPKGEPGSMGPRGENGVDGAPGPKGEPGHRGTDGAAGPRGAPGLKGEQGDTVVIDYDGRILDALKGPPGPQGPPGPPGIPGAKGELGLPGAPGIDGEKGPKGQKGDPGEPGPAGLKGEAGEMGLSGLPGADGLKGEKGESASDSLQESLAQLIVEPGPPGPPGPPGPMGLQGIQGPKGLDGAKGEKGASGERGPSGLPGPVGPPGLIGLPGTKGEKGRPGEPGLDGFPGPRGEKGDRSERGEKGERGVPGRKGVKGQKGEPGPPGLDQPCPVGPDGLPVPGCWHK